SEQUENCE LISTING (21 sequences)

SEQ ID NO: 1 - DNA, 1857 bp, Homo sapiens
gtaactatggatttcccctcttacaactttcaaaccagagttggagactcagcattggggttcgccctgcccgtagcacagccaagccctacctctcggttatcttttctcccgtcaccacccagtaaggtcatgtgcttccacccctggtcggatgtaacgctgccactcatgtcggtccctgagatccgggctgttgttgatgcatgggcctcagtcacagaggagctgggtgcccagtacccttgggtgcaggtttgtgaggtcgccccttcccctggatgggcagggagggggtgatgaagctttggttctggggagtaacatttctgtttccacagggtgtggtcaggagggagttgacttggtgtcttttggctaacagagctccgtatccctatctgatagatctttgaaaacaaaggtgccatgatgggctgttctaacccccacccccactgccaggtaagggtgtcaggggctccagtgggtttcttggctgagtctgagccagcactgtggacatgggaacaggattaatggatgggacagaggaaatatgccaatgatgtggaggcttggaggtaaaggacctgcctgttcttctctgcttttgccccttgacaggtatgggccagcagtttcctgccagatattgcccagcgtgaggagcgatctcagcaggcctataagagtcagcatggagagcccctgctaatggagtacagccgccaggagctactcaggaaggtgggagagagccaagccctgtgtccccaaggagtccctaactttcttatcccatgagagaggtgtgtaaaggagaaagctagaggtgaactagtagagagagacttgctaggaggccttagcaataatccagtaatctaaaggaaagatgatggtgacttagactcgggtggttagtggtagaggtggtgagaagacatcagatcctgggcacattcttttcttctgcttcccttgcctatttgctgaccacactccggctcctatgtcaccttgatgacttcctatccattctgtcttcctaggaacgtctggtcctaaccagtgagcactggttagtactggtccccttctgggcaacatggccctaccagacactgctgctgccccgtcggcatgtgcggcggctacctgagctgacccctgctgagcgtgatggtcagtctcccaagtaggatcctggggctaggcactggatggaggttgctcccagtagggtcagcatctggaccccaggctgagagtcaggctctgattccagatctagcctccatcatgaagaagctcttgaccaagtatgacaacctctttgagacgtcctttccctactccatgggctggcatggtgaggcttttcaagtacctatatttagccccaacaccatttctgggctcctgggctcagcctagtgaactgcaacctcaaaggagcaagccttgaaacagttgctgggggaagtggccagagtagagatgctgggactgagggtggagcagcaaacttggtgaaactacatctccaatgtgctttctaatctcctgccagctcttctcaagcaggggatcctgggagatgtagttttcagatacctggttgggtttgggagtaggtgctaacctggataactgtaaaagggctctctctccccactgtctctcttctttctgtcaggggctcccacaggatcagaggctggggccaactggaaccattggcagctgcacgctcattactaccctccgctcctgcgctctgccactgtccggaaattcatggttggctacgaaatgcttgctcaggctcagagggacctcacccctgagcag

SEQ ID NO: 2 - DNA, 22 bp, Homo sapiens
actgtaaaagggctctctctcc

SEQ ID NO: 3 - DNA, 19 bp, Homo sapiens
gcaagcatttcgtagccaa

SEQ ID NO: 4 - DNA, 31 bp, Homo sapiens
cgcaggagcggagggtagtaatgagcgtgca

SEQ ID NO: 5 - DNA, 20 bp, Homo sapiens
ctgccaatggtcccagttgg

SEQ ID NO: 6 - DNA, 21 bp, Homo sapiens
cttttggctaacagagctccg

SEQ ID NO: 7 - DNA, 21 bp, Homo sapiens
ttcccatgtccacagtgctgg

SEQ ID NO: 8 - DNA, 24 bp, Homo sapiens
gccaagaaacccactggagcccct

SEQ ID NO: 9 - DNA, 19 bp, Homo sapiens
acacccttacccggcagtg

SEQ ID NO: 10 - DNA, 21 bp, Homo sapiens
cacagccaagccctacctctc

SEQ ID NO: 11 - DNA, 21 bp, Homo sapiens
acctcacaaacctgcacccaa

SEQ ID NO: 12 - DNA, 30 bp, Homo sapiens
gaagcacatgaccttactgggtggtgacgg

SEQ ID NO: 13 - DNA, 20 bp, Homo sapiens
cgttacatccaaccaggggt

SEQ ID NO: 14 - DNA, 23 bp, Homo sapiens
gctgagagtcaggctctgattcc

SEQ ID NO: 15 - DNA, 20 bp, Homo sapiens
ccagaaatggtgttggggct

SEQ ID NO: 16 - DNA, 28 bp, Homo sapiens
ctttgagacgtcctttccctactccatg

SEQ ID NO: 17 - DNA, 22 bp, Homo sapiens
gctcttgaccaattatgacaac

SEQ ID NO: 18 - DNA, 20 bp, Homo sapiens
gaggcttggaggtaaaggac

SEQ ID NO: 19 - DNA, 20 bp, Homo sapiens
tccattagcaggggctctcc

SEQ ID NO: 20 - DNA, 28 bp, Homo sapiens
tgcccagcgtgaggagcgatctcagcag

SEQ ID NO: 21 - DNA, 20 bp, Homo sapiens
cagcagtttcccgccagata

(19) United States
(12) Patent Application Publication
(10) Pub. No.: US 2004/0005580 A1
(43) Pub. Date: Jan. 8, 2004

(54) Detecting mutations in the GALT gene by DNA melting curve analysis

(76) Inventors: Steven F. Dobrowolski, Park City, UT (US); Richard A. Banas, Turtle Creek, PA (US)

Correspondence Address: MCKAY & ASSOCIATES, PC., 801 MCNEILLY ROAD, PITTSBURG, PA, 15226, US

(21) Appl. No.: 10/283,443

(22) Filed: Oct. 30, 2002

Related U.S. Application Data
(60) Provisional application No. 60/335,630, filed on Oct. 31, 2001.

(51) Int. Cl. C12Q 1/68; C12P 19/34

(52) U.S. Cl. 435/6; 435/91.2

(57) ABSTRACT

A method is disclosed for detecting galactosemia-causing mutations in the GALT gene, comprising amplifying a portion of the GALT gene from isolated DNA and allowing a pair of labeled probes to hybridize to the portion. One of the labeled probes is adapted to match to a sequence that includes the galactosemia-causing mutation, and another of the labeled probes hybridizes to an adjacent sequence, thereby forming a hybrid. Melting curves of each hybrid are then analyzed, wherein peaks of the curves are produced at an acquired fluorescence and melting temperature, Tₘ; and a genotype is assigned based on the Tₘ of the hybrid. Resulting melting peaks are compared to reference sample peaks derived from samples characterized to contain the mutations, wherein the reference sample curves indicate a temperature change, ΔTₘ, between mutant and wild type peaks.

### SPECIFIC REFERENCE

The present application claims benefit of priority date so established by provisional application serial No. 60/335,630, filed Oct. 31, 2001.

### BACKGROUND

1. Field of the invention

The present invention relates generally to methods for detecting mutations in the galactose-1-phosphate uridyl transferase (GALT) gene. In particular, a set of methods are disclosed for genotyping specimens to determine their status (wild type or mutant) at five loci, each having a particular melting peak as they are subjected to melting curve analysis.

2. Description of the Related Art

Galactosemia is a standard component of most newborn metabolic screening programs. The classical form of galactosemia is caused by mutations in the galactose-1-phosphate uridyl-transferase (GALT) gene. Screening for galactosemia is achieved through analysis of total galactose (galactose and galactose-1-phosphate) and determining the activity of the GALT enzyme. This approach is effective, but environmental factors, specimen processing procedures, and the high frequency of the Duarte variant (N314D) necessitates further analysis to reduce false positive results.

Prospective screening of newborns for galactosemia is a routine procedure in the United States and many foreign countries. Screening utilizes the universally collected Guthrie dried blood card specimen (DBS) to assay for total galactose (galactose plus glactose-1-phosphate), and the activity of the galactose-1-phosphate uridyl transferase (GALT) enzyme. Total galactose is typically assayed for using NAD reduction analysis while GALT activity is determined using the Beutler assay.

Classical galactosemia results from mutations to the GALT gene, which cause severe perturbation in the activity of the corresponding enzyme. The GALT gene has been characterized and numerous mutations have been identified. Biochemical analysis for galactose and GALT activity are sound principle methods to prospectively assay newborns for galactosemia, however complicating factors can interfere with these results, necessitating further analysis.

It is commonly observed in newborn screening laboratories that environmental factors and sample collection/handling procedures, practiced at the site of specimen collection, may have severe adverse effects upon GALT activity, thereby causing abnormally low results in the Beutler assay. The most notable environmental influences upon GALT activity are heat and humidity. Specimens collected during hot, humid summer months, or in climates where such conditions are persistent, often present with reduced GALT activity in the Beutler assay. The practice of batching, where dried blood spot (DBS) specimens are permitted to accumulate before being mailed to the screening lab, also adversely affects GALT activity. Enzyme activity deteriorates over time, which minimizes the optimum time period between specimen collection and analysis. In addition steps must be taken to avoid the specimen's exposure to heat and humidity, which are the best practices to ensure optimal performance in the Beutler assay.

Additionally, reasons inherent to the screening process itself and the nature of mutations in the GALT gene necessitate analysis beyond the biochemical regimen. A false negative result may cause, at the minimum, serious medical consequences and, in a worse case scenario, death. Comparatively, a false positive result may lead to parental anxiety, mistrust of the screening process, and unnecessary medical procedures. To balance these concerns, screening labs require that critical result values fall within a range where false negative results are eliminated and false positive results are minimized.

The Duarte variant (N314D), carried by upwards of 5% of the general population, causes a partial loss (˜25%) of GALT activity. The high frequency of the Duarte variant is yet another complicating factor-in galactosemia screening.

Supplementing biochemical data using mutational analysis is a powerful method to reduce false positive results and may be used to provide unambiguous confirmation of true positive results. For example, a 2-tiered approach, biochemical analysis followed by gene-level analysis, is the standard employed by most newborn screening laboratories in their cystic fibrosis screening programs. Primary screening for cystic fibrosis is performed by analysis of circulating trypsinogen and those specimens having elevated trypsinogen are subsequently assayed for the CFTR Δ508 mutation. Delta F508 accounts for approximatelv 70% of CFTR mutations worldwide. The addition of Δ508 analysis has dramatically reduced the false positive rate in cystic fibrosis screening. A similar approach, as described herein, increases specificity in galactosemia screening.

Barriers to gene-level analysis in the screening lab include complexity and turnover time. Traditional methods for genetic analysis such as DNA sequencing, allele specific cleavage, and allele specific oligonucleotide hybridization are time consuming and labor intensive, thus limiting their usefulness in a high throughput laboratory. A recently developed platform dubbed the Lightcycler® eliminates essentially all issues of complexity, turnover time, and labor intensity encountered with classical methods of mutation analysis. The Lightcycler® utilizes rapid air driven thermal cycling and in-line fluorescence analysis of hybridization probes to generate melting curves, which are subsequently used to generate melting peaks for genotype assignment.

DNA melts at a defined temperature (Tₘ), wherein Tₘ is defined as the temperature at which half of the double helical structure is lost. This is generally the principle as known in the art supporting the Lightcycler®, since the melting temperature of a DNA molecule is dependent upon its nucleotide composition. DNA molecules rich in GC base pairs have a higher Tm than those having an abundance of AT base pairs. The Lightcycler® provides an innovative solution for identifying the base composition of a PCR amplified product, and in the present embodiment, allows for a qualitative method to assay for galactosemia-causing mutations.

### SUMMARY

Light Cycler technology and fluorescent-labeled hybridization probes are employed and a 5-mutation panel is described which includes the 4 most frequently encountered classical galactosemia alleles (Q188R, S135L, K285N, L195P) and the Duarte N314D variant. Five assays are performed simultaneously under a common set of conditions for both thermal cycling and melting curve analysis. Including DNA preparation, set-up, amplification, and analysis, the entire process requires less than 2 hours. These assays are useful to reduce false positive results, confirm classical galactosemia, and differentiate classical galactosemia from Duarte/Galactosemia (D/G) compound heterozygotes. These assays, owing to their speed and efficacy, are ideal for utilization in a high-throughput newborn screening laboratory.

Using the presented methodologies for melting curve analysis of galactosemia-causing mutations, genotype data is generated in minutes, as opposed to hours or even days required when using traditional methods. A second advantage to this platform is that it is a “closed tube” assay where both amplification and analysis are performed in a common reaction vessel. Unified amplification and analysis allows for simplified sample tracking and greatly reduces the likelihood of amplicon contamination in the laboratory.

Accordingly, what is provided is a method for detecting specific galactosemia-causing mutations in the GALT gene, comprising amplifying a portion of the GALT gene from isolated DNA, wherein said portion potentially contains the galactosemia-causing mutation, and thereby forming an amplification product; allowing a pair of labeled probes to hybridize to one strand of the amplification product, wherein one of the probes spans the allele of interest and can match to either the mutant or wild type allele and another of the labeled probes hybridizes to an adjacent sequence, thereby forming hybrids. The hybridized probes bring the donor fluorophore and acceptor fluorophore into close proximity allowing a fluorescent signal to be generated when the appropriate wavelength of light is provided. This fluorescent signal is used to generate melting curves. Genotype is assigned based on the Tₘ of the disassociated hybrid that forms the peak. The galactosemia-causing mutations include the four most frequently encountered classical galactosemia alleles (Q188R, S135L, K285N, L195P) and the Duarte N314D variant. Thus, the method further comprises the step of comparing the resulting melting curves to reference sample curves of samples characterized to contain the above mutations, wherein the reference sample curves indicate a temperature change, ΔTₘ, between mutant and wild type peaks.

### BRIEF DESCRIPTION OF THE DRAWINGS

FIG. 1 shows the portion of the sequence for each allele with the SNP, and the sequences of the detection, anchor probes used for hybridization, and the location of fluorescent moieties.

FIG. 2 shows the results of the melting peaks for each allele after simultaneous analysis. Melting peaks are well-separated, thereby facilitating unambiguous genotype assignment for each loci.

FIGS. 3A-E display the resulting melting peaks and acquired fluorescence for the N314D, Q188R, S135L, K285N, and L195P assays respectively.

### DETAILED DESCRIPTION OF THE PREFERRED EMBODIMENT

The mutations represented in the panel described herein represent the 4 most frequently encountered classical galactosemia mutations observed in the general United States population. Q188R is the most frequently encountered mutation representing approximately 70% of classical galactosemia alleles. The S135L mutation is most frequently observed among African Americans and is the second most frequently encountered allele. K285N is common in those of eastern European descent while L195P represents approximately 2% of classical galactosemia alleles. The Duarte variant, N314D, is present in approximately 5% of the US population and is probably the individually most complicating factor in screening for galactosemia. The so-called D/G compound heterozygotes (where a 314D allele is paired with a classical galactosemia mutation) may display both elevated total galactose and reduced GALT activity effectively mimicking classical galactosemia.

As used herein, “GALT” refers to the enzyme galactose-1-phosphate uridyltransferase, and “galactosemia” is the deficiency in the activity of the GALT enzyme. Screening for galactosemia is thus achieved through analysis of total galactose (galactose and galactose-1-phosphate) and determining the activity of the GALT enzyme.

For patient and/or reference specimen preparation, DNA samples are collected from any traditional methods, such as from any tissue or organ from which DNA can be amplified, or by purification from a dried blood spot on filter paper. “Reference specimens” as used herein, are previously characterized DNA specimens containing the mutations of interest. “Patient specimens” are the routinely collected DNA specimens that are to be screened and compared to results obtained from the reference specimens to determine a normal, mutant, or heterozygote sample.

The sequence of the human GALT gene (Genbank accession number M96264) is the basis for primer and probe design. A portion containing intron 4-exon 10 of the human GALT gene having the potential mutations or complementary positions is set forth in SEQ ID NO: 1 and is nucleotide region (1337-3193) of the above human GALT gene sequence.

A “primer” as used herein is a short piece of artificially made DNA complementary to a given DNA sequence and which acts as the initiation point from which replication proceeds via polymerase chain reaction (PCR). A “probe” is a fluorescent-labeled synthetic strand of DNA that anneals or “hybridizes” to a complementary DNA sequence generated by PCR. A “detection probe” hybridizes with a sequence that includes the site of the mutation and an “anchor probe” is another half of the probe set that hybridizes to an adjacent sequence. When both are hybridized it brings their respective fluorescent moieties into close proximity. A signal is generated by providing a specific wavelength of light, and fluorescence is monitored during incremental temperature increase to produce a melting curve. Melting curves are utilized to produce melting peaks. As will be further described, these resulting melting peaks are analyzed to determine wild type and mutant indications, wherein a temperature change, ΔTₘ, separates the different peaks. “A” as used in the claims may mean one or more depending on the context of the claim. It is not necessary to use only two probes as in the present assay. There are a few other types of probes that can produce these melting peaks without using two probes. For example, there are single probe systems with a single labeled probe that will produce melting peaks.

Primers, probes, the concentration at which each is used, and the associated SEQ ID NO are listed in table 1.

TABLE 1

| | Forward | | Reverse Primer | | Anchor | | Detection | |
|---|---|---|---|---|---|---|---|---|
| Allele | Primer | [ ] | Sequence | [ ] | Probe | [ ] | Probe | [ ] |
| | Sequence | μM | | μM | Sequence | μM | Sequence | μM |
| N314D | 5′ | 0.5 | 5′ | 0.3 | 5′ | 0.3 | 5′-LC Red 640 | 0.14 |
| | ACTGTAAAAG | | GCAAGCATTTCGT | | CGCAGGAGCGGAG | | CTGCCAATGGT | |
| | GGCTCTCTCT | | AGCCAA 3′ | | GGTAGTAATGAGC | | CCCAGTTGG-PO4 3′ | |
| | CC 3′ | | | | GTGCA-FITC 3′ | | | |
| SEQ ID | 2 | | 3 | | 4 | | 5 | |
| NO | | | | | | | | |
| Q188R | 5′ | 0.5 | 5′ | 0.5 | 5′ | 0.2 | 5′-LC Red 640 | 0.13 |
| | CTTTTGGCTA | | TTCCCATGTCCACA | | GCCAAGAAACCCA | | ACACCCTTACC | |
| | ACAGAGCTCC | | GTGCTGG 3′ | | CTGGAGCCCCT-FITC 3′ | | CGGCAGTG-PO4 3′ | |
| | G 3′ | | | | | | | |
| SEQ ID | 6 | | 7 | | 8 | | 9 | |
| NO | | | | | | | | |
| S135L | 5′ | 0.25 | 5′ | 0.125 | 5′-LC Red 640 | 0.1 | 5′ | 0.2 |
| | CACAGCCAAG | | ACCTCACAAACCT | | GAAGCACATGACC | | CGTTACATCCA | |
| | CCCTACCTCT | | GCACCCAA 3′ | | TTACTGGGTGGTG | | ACCAGGGGT-FITC 3′ | |
| | C3′ | | | | ACGG-PO4 3′ | | | |
| SEQ ID | 10 | | 11 | | 12 | | 13 | |
| NO | | | | | | | | |
| K285N | 5′ | 0.125 | 5′ | 0.25 | 5′-LC Red 640 | 0.2 | 5′ | 0.1 |
| | GCTGAGAGTC | | CCAGAAATGGTGT | | CTTTGAGACGTCCT | | GCTCTTGACCA | |
| | AGGCTCTGAT | | TGGGGCT 3′ | | TTCCCTACTCCATG-PO4 3′ | | ATTATGACAAC-FITC 3′ | |
| SEQ ID | 14 | | 15 | | 16 | | 17 | |
| NO | | | | | | | | |
| L195P | 5′ | 0.5 | 5′ | 0.5 | 5′-LC Red 640 | 0.2 | 5′ | 0.1 |
| | GAGGCTTGGA | | TCCATTAGCAGGG | | TGCCCAGCGTGAG | | CAGCAGTTTCC | |
| | GGTAAAGGAC | | GCTCTCC 3′ | | GAGCGATCTCAGC | | CGCCAGATA-FITC 3′ | |
| | 3′ | | | | AG-PO4 3′ | | | |
| SEQ ID | 18 | | 19 | | 21 | | 22 | |
| NO | | | | | | | | |

The number of nucleotides in the primers and probes may vary slightly. For a melting analysis assay, adequate thermal stability is needed such that the melting peak (Tₘ) is in a useful range, which may be defined generally as 50-70° C. As such, the Tₘ of the mismatched and matched probes should be within this range. The anchor probes serve to hold the second fluorophore in proximity to the first throughout the melting transition of the detection probe. As such, the anchor probe must have a higher Tm than the detection probe to which it is paired. Generally, at least 15% is required. In this preferred embodiment, 20-25% is allowed. So the length of the probes may vary depending upon the G/C content of the DNA to which it is hybridizing. If it is A/T rich, the probe is longer, and if it is G/C rich the probe may be shorter. Generally, this method starts with 30 nucleotides and nucleotides are either added or subtracted therefrom until a probe having desirable qualities is obtained. Such qualities include an adequately high melting temperature, no serious self hybridization or cross hybridization interactions, no serious self-interactions (folding) and no “false hybridization” within the amplified DNA fragment.

All primer pairs are designed to function under a common set of thermal cycling conditions. In this embodiment for example, all primer pairs are designed with Tm values between 59-64° C.

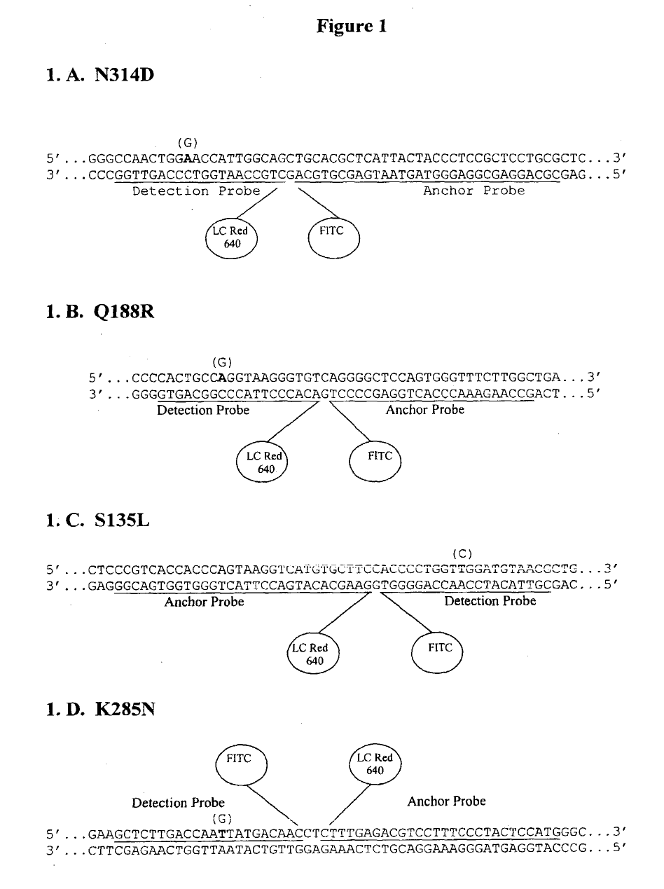

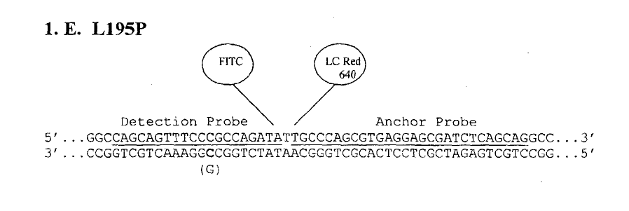

The organization of the anchor and detection probes for each allele is shown in FIG. 1. In this embodiment, one probe is labeled 3′ with FITC while the other probe is labeled 5′ with LC-red 640 and 3′ phosphorylated. Obviously, these labels can change if analysis takes place at a different wavelength. For example LC-red 705 could be used, which would change the interpretive guidelines as would be known in the art. Fluorescent-labeled probes are analyzed by spectrophotometry for oligonucleotide and fluorophore concentration. Probes with fluorophore/oligonucleotide ratios of 0.8-1.2 are generally suitable.

The amplicons, or PCR products, are preferably held to fewer than 200 base pairs. Though not necessary, this facilitates the optimum binding of hybridization probes. Table 2 shows the number of base pairs in the amplicons for the current assay.

TABLE 2

| Allele | PCR Product Length |
|---|---|
| N314D | 171 bp |
| Q188R | 160 bp |
| S135L | 190 bp |
| K285N | 155 bp |
| L195P | 149 bp |

Following amplification, the cycling protocol proceeds seamlessly to melting analysis. Fluorescence is acquired continuously during melting curve analysis and melting curves are constructed from data acquired during the upward temperature ramp.

The following example presents the recorded preparatory procedure and results obtained for specimen preparation, hybridization, and probe analysis.

### EXAMPLE

Specimens and DNA Preparation. Reference specimens, previously characterized to contain the mutations of interest, were utilized for assay development. Additional specimens were collected during routine newborn screening for galactosemia. Specimens whose reducing capacity were below 60 μM reduced NAD or whose total galactose was above 20 mg/dl were selected for mutation analysis. DNA was isolated from DBS specimens as previously described and 80-130 ηg is utilized as template in each reaction.

Amplification and Hybridization Probe Analysis. The sequence of the human GALT gene (Genbank accession number M96264) was used as a basis for primer and probe design. Primers and probes were designed in silico using Primer Premier 5.0 software. All primers and probes were HPLC purified and obtained from Operon technology (Alameda, Calif.). PCR reaction buffers, 20 mM MgCl₂ for the K285N and S135L assays, 30 mM MgCl₂ for the N314D, Q188R, and L195P assays, are obtained from Idaho Technology (Salt Lake City, Utah). All reactions use 0.6 U Klen taq DNA polymerase (AB Peptides, St. Louis, Mo.) complexed with TaqStart antibody (Clontech, Palo Alto, Calif.) according to manufacturers instructions. Primers, probes, and the concentration at which each is used are listed in table 1. The number of base pairs in the amplicons is listed in Table 2. All primer pairs were designed with Tm values between 59-64° C. and as such function under a common set of thermal cycling conditions. Amplification was performed in a Roche Light Cycler (Manheim, Germany). Cycling conditions were 40 cycles of 94° C., for 0 seconds (20°/second ramp speed)>60° C. for 20 seconds (20°/second ramp speed), >72° C., 0 seconds (2°/second ramp speed). Fluorescence was acquired at the end of the 20-second primer-annealing segment of the amplification. In the cases of N314D, S135L, and K285N assays, amplification was performed in an asymmetric fashion favoring the strand to which the hybridization probes bind (sense strand for S135L and N314D, antisense strand for K285N) while the Q188R and L195P assays were amplified in a symmetric manner. Amplicons were held to fewer than 200 base pairs (See Table 2), which facilitated optimum binding of hybridization probes. Following amplification, the cycling protocol proceeds seamlessly to melting analysis. Melting curve analysis used the following conditions: 97° C., 0 seconds, ramping at 2°/second down to 40° C., and ramping back up to 76° C. at 0.1° C/second. Fluorescence was acquired continuously during melting curve analysis and melting curves were constructed from data acquired during the upward ramp from 40° C. to 76° C.

The assays utilized “probe:probe” format for Light Cycler genotyping assays. The probe:probe format utilizes 2 oligonucleotide probes that hybridize to a selected strand of the amplicon. A detection probe was employed which matches a sequence that includes the site of the mutation and an anchor probe, which hybridizes to an adjacent sequence. In these assays there is a 1-nucleotide gap between the anchor and detection probes. One probe was labeled 3′ with FITC while the other probe was labeled 5′ with LC-red 640 and 3′ phosphorylated. Probes were designed to maximize destabilization of the mismatch hybrid and it was determined in all 5 instances that matching the mutant allele with subsequent mismatch to the wild type allele provided the most effective probe design.

Results

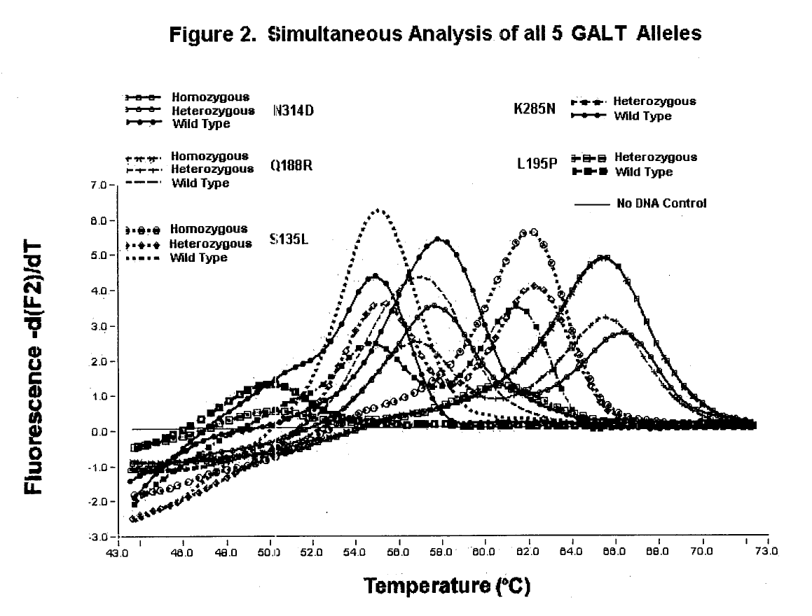

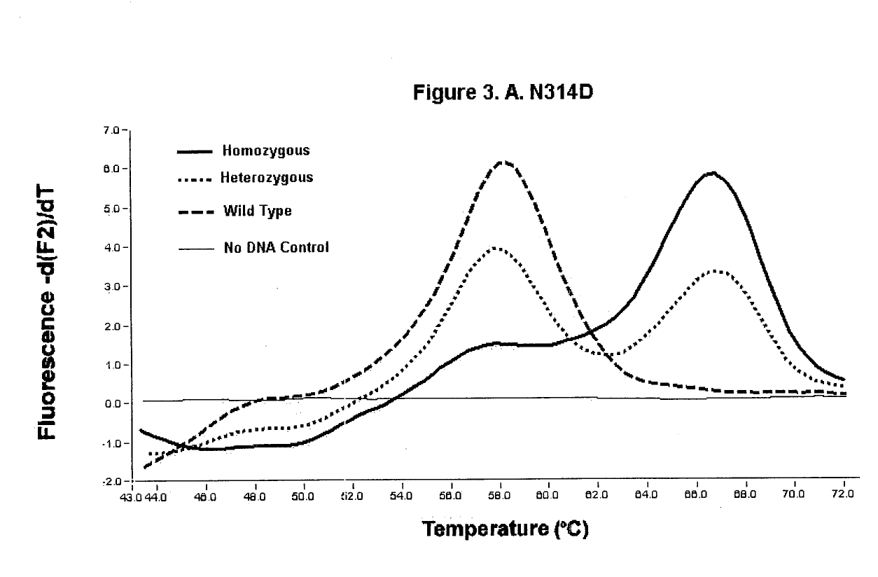

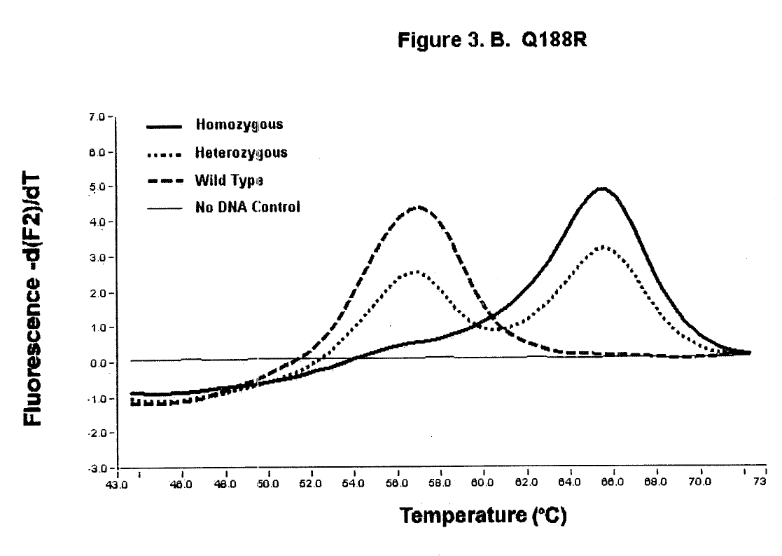

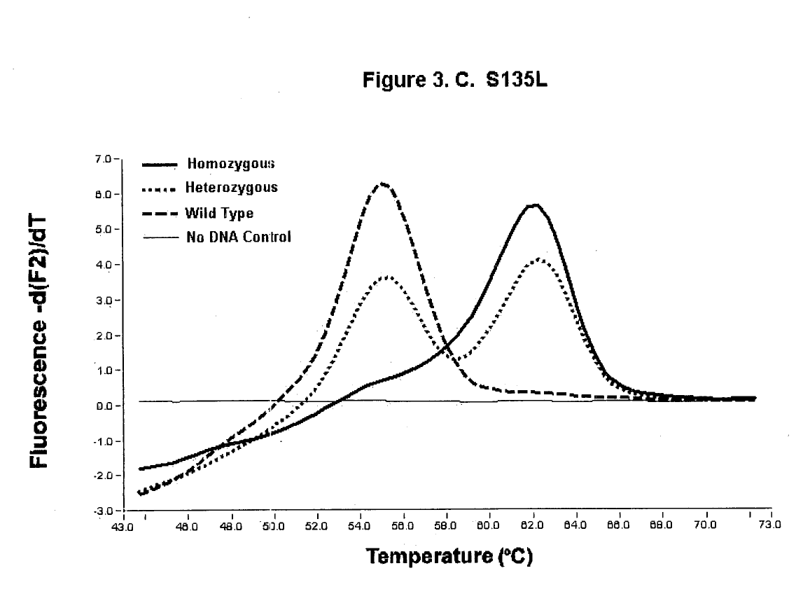

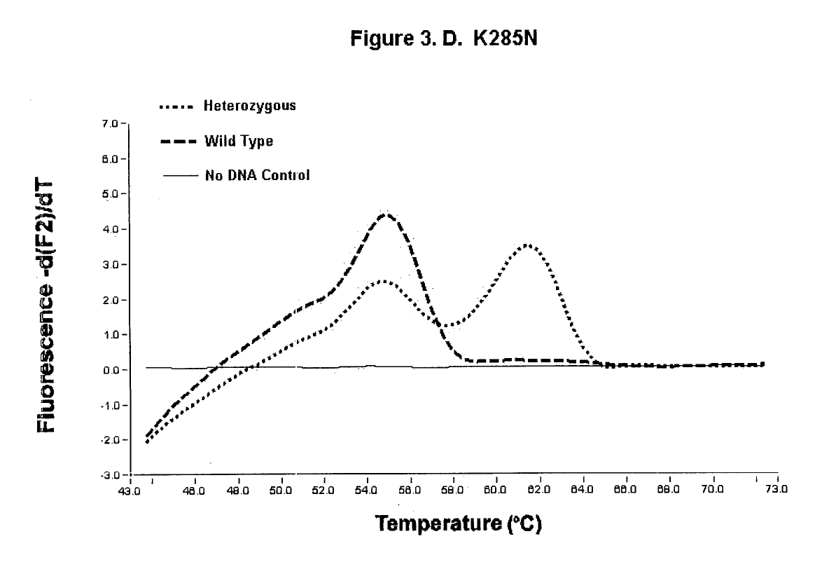

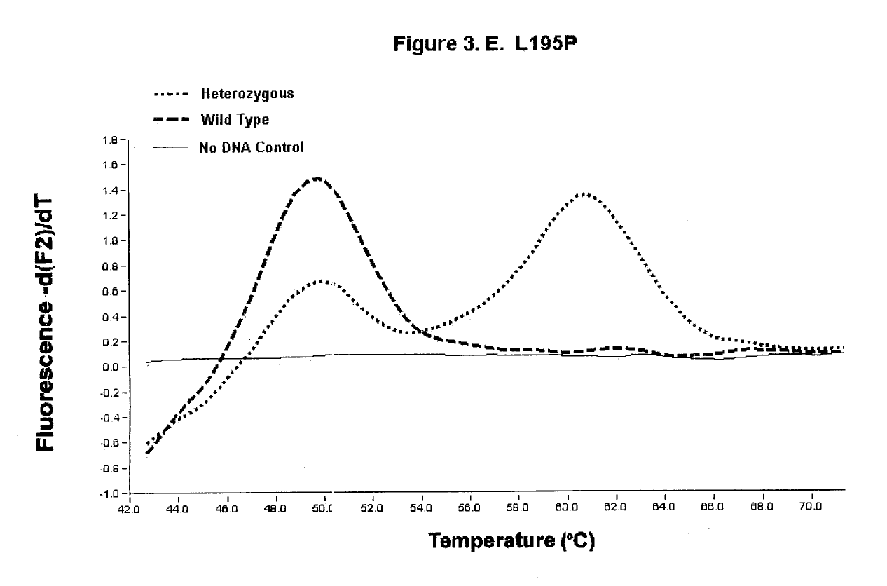

FIG. 2 display all 5 assays analyzed simultaneously as would be initially observed following routine analysis. The N314D, Q188R, and S135L assays display analysis of specimens that are homozygous wild type, homozygous mutant, heterozygous, and a no-amplification control. Assays for K285N and L195P show the analysis of specimens that are homozygous wild type, heterozygous, and no amplification control. Combined analysis as shown in FIG. 2 is complex, so individual assays are subsequently viewed by selecting individual specimens or groups of specimens (e.g. control and test specimens) as seen in FIGS. 3A-E.

FIGS. 3A-E display melting peaks for the N314D, Q188R, S135L, K285N, and L195P assays respectively. In all cases the peak representing the mutant allele is high temperature melting peak (perfect match with the detection probe) while the wild type allele is the low-temperature melting peak (mismatch hybrid with the detection probe). Melting peaks are well separated facilitating unambiguous genotype assignment for each loci. The melting temperature of each peak and the ΔTₘ separating the wild type and mutant peaks for individual assays are displayed in Table 3.

TABLE 3

| Allele | Wild Type Tm | Mutant Tm | ΔTm |
|---|---|---|---|
| N314D | 57.83 | 66.16 | 8.33 |
| Q188R | 56.80 | 65.47 | 8.67 |
| S135L | 55.08 | 62.09 | 7.01 |
| K285N | 54.89 | 61.41 | 6.52 |
| L195P | 49.94 | 60.57 | 10.63 |

Peak separation ranges from 6.52°-10.630° C., enabling easy and unambiguous genotype assignment.

I claim:

1. A method for detecting galactosemia-causing mutations in a GALT gene, comprising:
amplifying a portion of said GALT gene from isolated patient DNA, thereby forming an amplification product;
allowing a pair of labeled probes to hybridize to one strand of said amplification product, wherein a detection probe is adapted to match to a sequence that may include said galactosemia-causing mutation, and an anchor probe hybridizes to an adjacent sequence, thereby forming hybrids;
generating a melting curve having peaks indicative of the melting temperature (Tm) of each of said hybrids;
determining a temperature change, ΔTₘ, between a wild type peak and any mutant peak of said melting curve at each loci of said hybrid.

2. The method of claim 1, wherein said galactosemia-causing mutations are selected from the group consisting of Q188R, S135L, K285N, L195P and N314D.

3. The method of claim 1, wherein for the step of amplifying a portion of said GALT gene from isolated patient DNA, primer pairs having Tₘ values between 59° C. and 64° C. are used.

4. The method of claim 1, further comprising the step of assigning a genotype to said patient DNA.

5. The method of claim 1, wherein said amplification product is held to fewer than 200 base pairs.

6. The method of claim 1, wherein said detection probe is selected from the group consisting of those such sequences as set forth in SEQ ID NO: 5, SEQ ID NO: 9, SEQ ID NO: 13, SEQ ID NO: 17, and SEQ ID NO: 22.

7. The method of claim 1, wherein said anchor probe is selected from the group consisting of those such sequences as set forth in SEQ ID NO: 4, SEQ ID NO: 8, SEQ ID NO: 12, SEQ ID NO: 16, and SEQ ID NO: 21.

8. A method for detecting galactosemia-causing mutations in a GALT gene, comprising:
amplifying a portion of said GALT gene from isolated patient DNA, thereby forming an amplification product;
allowing a pair of labeled probes to hybridize to one strand of said amplification product, wherein a detection probe selected from the group consisting of those such sequences as set forth in SEQ ID NO: 5, SEQ ID NO: 9, SEQ ID NO: 13, SEQ ID NO: 17, and SEQ ID NO: 22 is allowed to match to a sequence that may include said galactosemia-causing mutation, and an anchor probe selected from the group consisting of those such sequences as set forth in SEQ ID NO: 4, SEQ ID NO: 8, SEQ ID NO: 12, SEQ ID NO: 16, and SEQ ID NO: 21 is allowed to hybridize to an adjacent sequence, thereby forming hybrids; and,
generating a melting curve having peaks indicative of the melting temperature (Tm) of each of said hybrids.

9. The method of claim 8, wherein said galactosemia-causing mutations are selected from the group consisting of Q188R, S135L, K285N, L195P and N314D.

10. The method of claim 8, wherein for the step of amplifying a portion of said GALT gene from isolated patient DNA, primer pairs having Tₘ values between 59° C. and 64° C. are used.

11. The method of claim 8, further comprising the step of assigning a genotype to said patient DNA.

12. The method of claim 8, wherein said amplification product is held to fewer than 200 base pairs.

13. A labeled probe for use in a method for detecting galactosemia-causing mutations in a GALT gene, comprising:
a detection probe selected from the group consisting of those such sequences as set forth in SEQ ID NO: 5, SEQ ID NO: 9, SEQ ID NO: 13, SEQ ID NO: 17, and SEQ ID NO: 22.

14. The probe as claimed in claim 13, wherein said detection probe is labeled with LC Red 640.

15. The probe as claimed in claim 14, wherein said detection probe is phosphorylated.

16. The probe as claimed in claim 13, wherein said detection probe is labeled with FITC.

17. A labeled probe for use in a method for detecting galactosemia-causing mutations in a GALT gene, comprising:
an anchor probe selected from the group consisting of those such sequences as set forth in SEQ ID NO: 4, SEQ ID NO: 8, SEQ ID NO: 12, SEQ ID NO: 16, and SEQ ID NO: 21.

18. The probe as claimed in claim 17, wherein said anchor probe is labeled with LC Red 640.

19. The probe as claimed in claim 18, wherein said anchor probe is phosphorylated.

20. The probe as claimed in claim 17, wherein said anchor probe is labeled with FITC.
